(19) United States
(12) Patent Application Publication
(10) Pub. No.: US 2023/0061628 A1
(43) Pub. Date: Mar. 2, 2023

(54) OPHTHALMOLOGIC MICROSCOPE WITH SYNCHRONIZED LIGHT SOURCE AND CAMERA

(71) Applicant: Haag-Streit AG, Köniz (CH)

(72) Inventors: Caspar TRITTIBACH, Bern (CH); Frank ZUMKEHR, Zollikofen (CH)

(73) Assignee: Haag-Streit AG, Köniz (CH)

(21) Appl. No.: 17/792,038

(22) PCT Filed: Jan. 13, 2020
(86) PCT No.: PCT/EP2020/050728

§ 371 Date: Jul. 11, 2022

(51) Int. Cl. A61B 3/14; A61B 3/135

(52) U.S. Cl. CPC A61B 3/14; A61B 3/135; A61B 2560/0223

(57) ABSTRACT

The ophthalmologic microscope has an illumination device for projecting light onto an eye to be observed and a microscope device with a camera to view the eye. The illumination device generates pulsed light and is slaved to the frame rate of the camera, which allows to run the camera in free-running mode for achieving a high frame rate. The light is pulsed at least at twice the frame rate of the camera to reduce flicker. The illumination device uses an array of micro-mirrors as spatial light modulator, and the mirrors are controlled for a balanced deflection over the frame cycles.

### TECHNICAL FIELD

The invention relates to ophthalmologic microscopes, in particular slit lamp microscopes, having an illumination device adapted to generate illumination pulses on the eye and a microscope device with a camera for recording images of the eye. The invention also relates to methods for operating such a device.

### BACKGROUND ART

EP 2549913 describes an ophthalmologic microscope having an illumination device generating illumination pulses as well as a microscope having a camera. The pulsed light and the camera are synchronized in order to have a defined illumination while recording a camera frame.

### DISCLOSURE OF THE INVENTION

The problem to be solved by the present invention is to provide an improved control for such a microscope.

This problem is solved by the devices and methods according to the independent claims.

Accordingly, the invention relates to ophthalmologic microscopes comprising at least the following elements:

i) An illumination device generating illumination pulses: The illumination device is adapted and structured to generate individual light pulses for illuminating the eye to be examined. It comprises at least the following elements:
a) At least one light source: This is the part of the illumination device that generates the light.
b) A spatial light modulator: This part is structured to spatially modulate the light. It may e.g. be a static slit, but advantageously it is an electronically controlled light modulator.
c) Illumination imaging optics: This part is structured to project the light onto the eye. Advantageously, it projects an image of the spatial light modulator onto the eye, thereby generating a defined illumination pattern on the eye.

ii) A microscope device: The microscope device is adapted to record an image of the eye. It comprises at least the following elements:
a) Microscope optics: The microscope optics generates an image of the eye.
b) At least one electronic camera: The camera is adapted to record the projected image from the microscope optics.

iii) A control unit: The control unit controls the operation of the elements of the device.

According to a first aspect of the invention, the camera has a frame signal output carrying a signal indicative of the times when the camera records a frame. In particular, it may generate a single pulse synchronized to each frame it takes, i.e. synchronized to the integration phase of that frame.

The control unit is structured to synchronize the illumination device to this frame signal output. This allows to run the camera independently, e.g. in its native free-running mode, which allows for a faster frame rate. Hence, in this configuration, the camera acts master device while the illumination device acts as a slave device synchronized to the timing of the master device.

In a second aspect of the invention, the control unit is structured to generate at least two illumination pulses on the eye for each frame recorded by the camera. This has the advantage that the light pulse rate that the eye sees is higher than the frame rate of the camera, thereby reducing the risk of irritating the patient with flicker effects.

The invention also relates to methods for operating ophthalmologic microscopes, in particular the microscopes as mentioned above. Same as above, the microscope comprises at least the following elements:

i) An illumination device generating illumination pulses: The illumination device is adapted and structured to generate individual light pulses for illuminating the eye to be examined. It comprises at least the following elements:
a) At least one light source: This is the part of the illumination device that generates the light.
b) A spatial light modulator: This part is structured to spatially modulate the light. It may e.g. be a static slit, but advantageously it is an electronically controlled light modulator.
c) Illumination imaging optics: This part is structured to project the light onto the eye. Advantageously, it projects an image of the spatial light modulator onto the eye, thereby generating a defined illumination pattern on the eye.

ii) A microscope device: The microscope device is adapted to record an image of the eye. It comprises at least the following elements:
a) Microscope optics: The microscope optics generates an image of the eye.
b) At least one electronic camera: The camera is adapted to record the projected image from the microscope optics.

iii) A control unit: The control unit controls the operation of the elements of the device.

The method comprises the step of sequentially recording frames by means of the camera.

According to the first aspect of the invention, the method comprises the steps of generating, by means of the illumination device, light pulses synchronized to the frame signal output of the camera.

According to the second aspect of the invention, the method further comprises the step of generating, by means of the illumination device, at least two illumination pulses for each frame recorded by the camera.

The two aspects of the invention can be used separately or in combination.

Advantageously, the camera is run in its “free-running mode”. In the free-running mode, the camera is not recording its frames synchronized with an external trigger signal but rather records a next frame as soon as it has finished recording and processing the previous frame. In free-running mode, cameras can reach a higher frame rate than when they are triggered to record the frames by means of an external source.

Advantageously, the spatial light modulator is an electronically controlled spatial light modulator comprising a two-dimensional array of individually controllable pixels, which may be synchronized, by the control unit of the device, to the frame signal output of the camera.

In one embodiment, the spatial light modulator may comprise a two-dimensional array of micro-mirrors that are individually deflectable into a first and a second position. The first position may e.g. correspond to the on-state and the second position may e.g. correspond to the off-state of the pixel. In this context, a micro-mirror is advantageously a mirror having an area of less than 1 mm², in particular less than 100 μm².

The light source is advantageously pulsed, i.e. the control unit is structured to pulse the light source. Advantageously, this is done in a manner synchronized to the frame signal output of the camera.

If the microscope uses a pulsed illumination as well as a light modulator having an array of individually controllable pixels, the control unit may be adapted to:
bringing the pixels into a given configuration during a dark phase prior to a given light pulse, and
starting the light pulse only when the pixels are in the desired configuration.

In this case, the dark phase is before a light pulse is exploited for setting up the light modulator. This setting up may take some time, e.g. because the pixels are slow or because it takes time to feed the pattern information sequentially into the light modulator, and it is therefore best done before the light pulse.

The method or control unit may be adapted to perform at least the following two steps:
Predicting the time when the camera will record a next frame: This prediction is made depending on the time when the camera recorded at least one previous frame. In particular, it may depend on the times when the camera recorded several previous frames.
Preparing at least part of the illumination device before the predicted time for recording the next frame.

This allows to set up at least part of the illumination optics, e.g. the spatial light modulator, before a frame is recorded, thereby allowing for a more defined image recording. This is particularly advantageous in the first aspect of the invention.

The device may further comprise several light sources and at least one light detector adapted to measure the light intensity at a location between the light sources and the spatial light modulator, i.e. the brightness of the light source independently of the state of the light modulator. In that case, the method or control unit may be adapted to perform at least the following two steps:
Bringing the light modulator into a non-transmitting mode: In this mode, no light is projected onto the eye even if the light source is switched on.
While the light modulator is in this non-transmitting mode, pulsing one light source for measuring the brightness of this light source.

This allows to intermittently—in particular while recording of a series of image frames—control the current brightness of the light source. This information can then be used e.g. for adjusting the current through the light source during light pulses while recording the frames on the on-time of the pixels of the light modulator while recording the frames in order to achieve a defined illumination.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows an embodiment of an ophthalmologic microscope,

FIG. 2 shows an embodiment of the components of the illumination device,

FIG. 3 is a schematic representation of micro-mirror-based spatial light modulator, and

FIG. 4 shows an embodiment of a timing diagram for various components of the device.

### MODES FOR CARRYING OUT THE INVENTION

### Overview

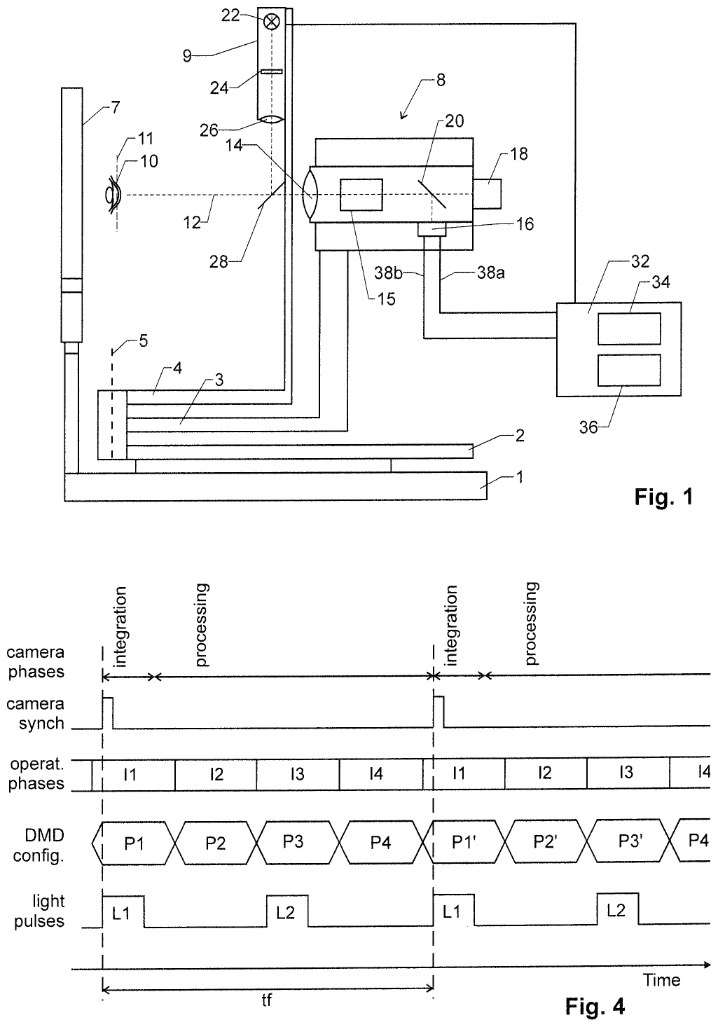

FIG. 1 shows an embodiment of an ophthalmologic microscope, in particular a slit lamp microscope.

The microscope has a base 1 resting e.g. on a desk, a translationally displaceable stage 2 mounted to base 1, a first arm 3, and a second arm 4.

Stage 2 can be linearly displaced along horizontal directions x and z in respect to base 1.

The arms 3 and 4 are mounted to stage 2 and pivotal about a common vertical pivot axis 5, i.e. an axis parallel to vertical direction y.

The device may further include a headrest 7 mounted to base 1 for receiving the patient's head.

Arm 3 carries a microscope device 8, and arm 4 carries an illumination device 9, such as a slit lamp.

Microscope device 8 has an optical axis 12. It comprises microscope optics 14, 15, such as an objective 14 and zoom optics 15, which project an image of eye 10 onto a camera 16 and, optionally, an eyepiece 18. A beam splitter 20 may be provided to spilt light between these components.

Illumination device 9 is adapted to project a structured light beam onto the eye 10 to be examined. It comprises a light source 22, a spatial light modulator 24, and illumination imaging optics 26.

Light source 22 can e.g. comprise several units emitting different wavelengths, e.g. in the red, green, blue, and infrared range of the optical spectrum. These units can be controlled separately in order to change the color of light source 22.

Advantageously, the light source 22 comprises at least one LED and/or semiconductor laser. This type of light sources are advantageous because LEDs and semiconductor lasers can be pulsed rapidly and precisely.

Illumination imaging optics 26 projects the light from modulator 24 onto e.g. the anterior surface of eye 10, e.g. via a mirror 28 mounted to arm 4. The anterior surface of eye 10 is assumed to be located at a target plane 11, which is the optically conjugate plane of spatial light modulator 24 in respect to illumination imaging optics 26.

Illumination device 9 can be arranged above or below mirror 28.

A control unit 32 controls the components of the microscope. In particular, it may e.g. comprise a microprocessor 34 and a memory 36. Microprocessor 34 is programmed to carry out the steps of the method as described below, and memory 36 contains the data and/or instructions to do so.

Camera 16 has is adapted to record a series of individual frames of the image projected onto it.

It has a frame signal output 38a carrying a signal indicative of the time when a frame is recorded. It also has a data output 38b for transferring the recorded image. Both outputs 38a, 38b may be connected to control unit 32.

Camera 16 may e.g. by a CCD camera or any other semiconductorbased camera, such as a camera of the IMX series by Sony.

In semiconductor cameras, the process of recording a frame involves the integration of the optical signal over a certain period. At the end of the integration period, the image can be read out.

The signal at frame signal output 38a is in synch with this integration phase and, depending on camera type, it may e.g. be issued at the beginning or the end of the integration phase.

Illumination Device

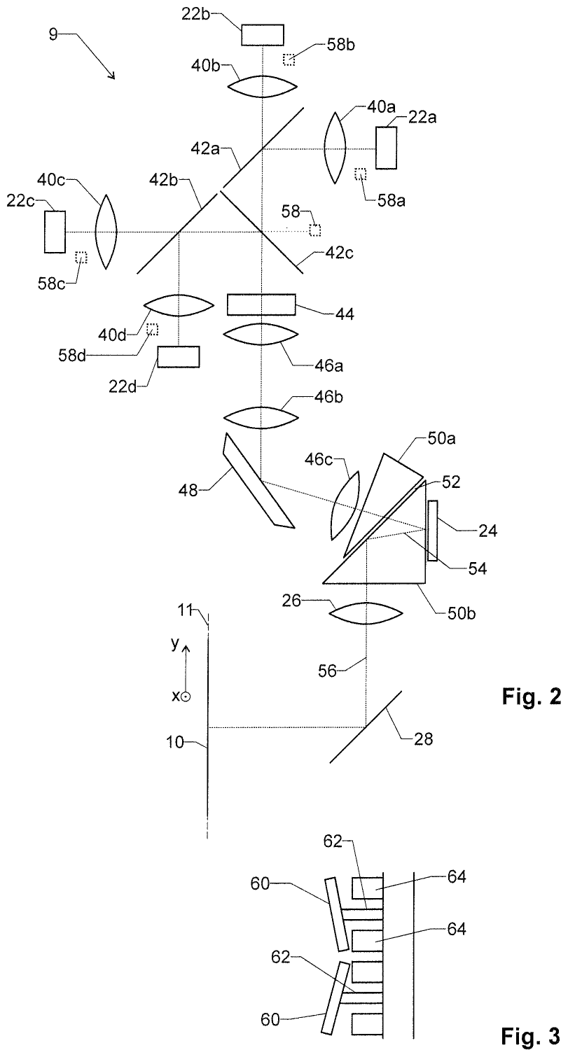

FIG. 2 shows a more detailed embodiment of illumination device 9. It is designed to project an illumination field of defined, sharp contours onto target, such as the eye 10. The illumination field may e.g. be round, rectangular, or slit-shaped. Even though the illumination device is called a “slit-lamp” herein, the illumination field does not need to be slit-shaped at all. It can take any shape.

In the present embodiment, illumination device 9 comprises four light sources 22a-22d of different spectral emission characteristics. For example, they may include an infrared light source, a red light source, a green light source, and a blue light source. Advantageously, the light sources are LEDs. In particular, each light source may be a single LED.

The light from each light source is substantially collimated by means of collimation optics 40a-40d.

Three dichroic mirrors 42a, 42b, 42c are used to combine the light from the light sources 22a-22d to become coaxial.

The combined light is passed through homogenization optics 44, such as a fly-eye lens array, e.g. as described in U.S. Pat. No. 6,507,434.

Two cylindrical lenses 46a, 46b, a further lens 46c, and the homogenization optics 44 together also widen the light beam along one direction, e.g. giving it an elongate cross-section, e.g. having a width-to-height ratio of 16:9, for better matching the typically available form factor of spatial light modulators.

A mirror 48 deflects the light into an assembly of two prisms 50a, 50b with a gap 52 between them.

The light beam passes prism 50a, gap 52, and prism 50b and arrives at spatial light modulator 24.

In the shown embodiment, spatial light modulator 24 is a DMD (“digital micro-mirror device”) with a two-dimensional array of individually deflectable micro-mirrors. Control unit 32 is adapted to control the alignment of each micro-mirror, e.g. between a first and a second position.

FIG. 3 shows, schematically, a sectional view of two micro-mirrors 60 of such a DMD. Each mirror 60 is held by an elastic support 62 and deflectable by means of electrodes 64. Devices of this type are known to the skilled person.

For the micro-mirrors 60 being in the first position, the light is reflected back into prism 50b along a direction denoted by 54 in FIG. 2. Light traveling along this direction 54 is subject to total internal reflection at the interface of second prism 50b to gap 52 and reflected into a direction denoted by 56 in FIG. 2.

For the micro-mirrors 60 being in the second position, the light is still reflected back into prism 50b, but along a different direction (not shown in FIG. 2), along which it does not fulfil the conditions for total internal reflection at the interface to gap 52. The small fraction still reflected at this interface will travel in a direction different from direction 56 and not be further processed by illumination imaging optics 26 described in the following.

Hence, control unit 32 is able to individually set each pixel (each micro-mirror 60) of spatial light modulator 24 into an on-state and an off-state, thereby defining the contour and shape of the light field at target 10 (which is assumed to be located in the target plane 11 of the illumination device).

The light from the pixels of spatial light modulator 24 enters illumination imaging optics 26, which may include one or more lenses. From there, it may pass mirror 28 to arrive at target 10.

The illumination imaging optics images spatial light modulator 24 onto target 10, i.e. target 10 is at the target plane 11, which is the conjugate plane, in respect to illumination imaging optics 26, of the plane 62 of spatial light modulator 24.

Operation

The process of recording a series of frames by means of camera 16 is illustrated in FIG. 4.

As can be seen in the first line of FIG. 4, the camera runs through integration and processing phases, which may also at least partially overlap.

In each integration phase, the pixels of the camera integrate the light impinging on them. Then, the respective signal is locked, converted to a digital value, and read out by control unit 32 during a processing phase. This process repeats itself in repetitive frame cycles.

Advantageously, camera 16 is operated in free-running mode, i.e. it repetitively records frames without waiting for external trigger signals. This allows to operate camera 16 at a high frame rate.

Each integration phase is marked by a camera synch signal, as shown in the second line of FIG. 4. If camera 16 is operating in free-running mode, this is the signal on frame signal output 38a.

Depending on the specific hardware used, the signal “camera synch” may e.g. coincide with the beginning or the end of the integration phase.

Control unit 32 operates spatial light modulator 24 and light source 22 in synchronicity with the “camera synch” signal as shown in the third to fifth lines of FIG. 4.

In the shown embodiment, control unit 32 operates illumination device 9, during one frame cycle, in four operating phases I1, I2, I3, and I4. They are repeated in each frame cycle. The integration phase of the camera falls into operating phase I1, while the operating phases I2-I4 are outside the integration phase.

In operating phase I1, spatial light modulator 24 is configured for the appropriate configuration (pattern) P1 of pixels. Advantageously, this configuration is completely set up before the integration phase starts (see fourth line of FIG. 4).

Only then, light source 22 is switched on (see fifth line of FIG. 4) to start a first light pulse L1.

In the shown example, configuration P1 is maintained over the whole integration phase. Also, the light source 22 remains switched on over the whole integration phase (light pulse L1).

When the integration phase has ended, light source 22 is switched off.

The integration phase may be shorter than first operating phase I1.

In operating phase I2, spatial light modulator 24 is brought into a second configuration (pattern) P2 while light source 22 remains switched off.

In operating phase I3, spatial light modulator 24 is brought into a third configuration (pattern) P3, and light source 22 is switched on again in order to generate a light pulse L2, which is switched off again before or at the end of operating phase I3.

In operating phase I4, spatial light modulator 24 is set to a fourth configuration (pattern) P4 and light source 22 remains switched off.

Then, the process repeats with phases I1-I4 and with possible different configurations P1′, P2′, P3′, P4′.

In one embodiment, the configurations are as follows:
Configuration P1 corresponds to the illumination pattern that is desired during recording of the respective frame. It may remain unchanged over the whole integration phase. Alternatively, however, it may be changed during the integration phase, e.g. by switching individual pixels from their on-state to their off-state in order to individually decrease the illumination brightness of said pixels.
Configuration P3 advantageously corresponds to configuration P1, i.e. it has the same pixels switched on and off as configuration P1. This generates two identical illumination patterns on the eye at twice the framerate, thereby reducing flicker as described above.
Configurations P2 and P4 are advantageously the “opposite” of configurations P1 and P3, i.e. when a pixel is on in configuration P1 or P3, the same pixel is off in configuration P2 and P4, and vice versa. This is based on the understanding that the time to failure of DMD spatial light modulators can be increased by regularly bringing each mirror into both its positions for similar periods of time. The present embodiment uses the dark phases between the light pulses L1, L2 to do that. Advantageously, the operating phases I1-I4 have equal or similar lengths in order to compensate the strain in the supports 62 of the mirrors 60 of spatial light modulator 24. Advantageously, the longest one of the operating phases I1-I4 is no more than 10 times, in particular no more than five times, in particular no more than 3 times, as long as the shortest one of the operating phases I1-I4.

The scheme of FIG. 4 achieves two goals:

1. It balances the mirror deflection in spatial light modulator 24, thereby reducing aging of the modulator.

2. It allows to generate light pulses at the double framerate of camera 16, thereby reducing flicker.

In order to achieve goal 1, in more general terms, control unit 32 may be configured to bring, over one frame cycle (i.e. over one cycle of the integration and processing phase) of camera 16, i.e. for each frame, each micro-mirror 60 of spatial light modulator 24 into its first position during a time t1 and into its second, opposite position during a time t2, where t1 and t2 are comparable, i.e. where

0.1<t1/t2<10, (1)

in particular wherein

0.2<t1/t2<5, (2)

and in particular wherein

0.33<t1/t2<3. (3)

In order to achieve goal 2, in more general terms, control unit 32 may be configured to generate, over one frame cycle of camera 16, i.e. for each frame, at least two separate light pulses, wherein one of the light pulses falls into the integration phase of camera 16 while the other light pulse(s) falls outside the integration phase.

Advantageously, the light pulses L1, L2 have equal durations (in particular within 10%) and, for all light pulses, spatial light modulator 24 has the same configuration (pattern), and the light pulses are separated by dark phases of equal length (i.e. the dark phases have equal length, in particular within 10%). This further reduces flicker.

Advantageously, the repetition rate of the light pulses is at least 70 Hz.

If goal 1 is not required (e.g. because a spatial light modulator not sensitive to unbalanced pixel settings is used, e.g. an LCD modulator), the configuration of spatial light modulator 24 may be any pattern during operating phases I2 and I4.

If goal 2 is not required (e.g. because the frame rate of camera 16 is inherently high enough to avoid flicker effects), operating phases I3 and I4 may be omitted.

If goals 1 and 2 are both to be achieved, control unit 32 is advantageously adapted to bring spatial light modulator 24, between the light pulses L1, L2, into configurations P2 and P4 opposite to the configurations P1 and P3 during the light pulses. In this context, “opposite” first and second configurations means that that if, in the first configuration, a given pixel is in its first state during a time ta and its second state during a time tb, in the second configuration it will be (within an accuracy of 10% or better) in its first state during time tb and its second state during time ta.

If the process shown in FIG. 4 is to be synchronized to frame signal output 38a, the operating phases I1-I4 should be synchronized to the integration phases of camera 16, such that each integration phase falls into a defined position of first operating phase I1.

To do so, control unit 32 is advantageously adapted to predict the time when camera 16 will record a next frame because, for example, it may need to configure spatial light modulator 24 before the integration phase of camera 16 starts, i.e. in the embodiment of FIG. 4 before the camera synch pulse arrives.

For this purpose, control unit 32 may e.g. use an estimate of the time tf between two consecutive frames.

This estimate for time tf can e.g. be a value stored in control unit 32 by the manufacturer. Alternatively, control unit 32 can measure it during operation, i.e. it can determine it from the times when camera 16 recorded previous frames.

Then, depending on the time when camera 16 recorded the last frame, i.e. depending on the time of the last camera synch pulse, the time of the next camera synch pulse, i.e. of the next integration phase, can be predicted.

This allows control unit 32 to prepare at least part of illumination device 9, such as spatial light modulator 34 and/or light source 22, before the predicted time of the next integration phase.

Position Balancing of the Micro-Mirrors

As mentioned above in reference to relation (1), (2), or (3), the positions of the micro-mirrors 60 are advantageously balanced, at least to some degree, in order to reduce mechanical strain.

This balancing takes place over no more than N consecutive frame cycles of the camera, i.e. it may not be fulfilled for each single frame cycle, but over the total times t1 and t2 over of a larger number N of frame cycles, with N≤100, may fulfill the relations (1), (2), or (3).

Advantageously, though, relation (1), (2), or (3) are fulfilled for a smaller number of cycles, such as N≤10 in order to reduce asymmetric strain further. In a most advantageous embodiment, N=1, i.e. balancing in the sense of relation (1), (2), or (3) takes place over each single frame cycle. This greatly simplifies the control of the device because it obviates the need to “plan” the mirror positions over two or more frame cycles.

In the period where the light source is operating and the camera is integrating, the position of each micro-mirror 60 is given by the amount of light to be generated for the respective pixel. Hence, balancing in order to fulfill relation (1), (2), or (3) advantageously takes place during the time when at least one of the following conditions is met:
the camera is not integrating and/or
the light source is not lit.

Advantageously, though, the balancing takes place when the light source is not lit, thereby reducing the risk of unwanted flicker in the patient's eye.

Note that t1 and t2 are typically different for at least some of the micro-mirrors, i.e. t1 and t2 are, in a most general case, dependent on the location i, j of the mirror in the modulator, i.e. t1(i,j) may differ from t1(i′,j′) if i≠i′ and/or j≠j′.

For example, one pixel may be kept dark while integrating the image, and another pixel may be kept bright while integrating the image. It may even be that a third pixel may be kept bright over a first part of the integration and switched to dark for a second part of the integration in order to achieve a pixel-wise brightness-modulation. In these cases, the times t1 and t2 may be different for both or all three types of pixels.

The present technique is particularly important if there is only a single light source or if there are several light sourced that are, however, switched on in an overlapping mater, i.e. if there is at least one time in the frame cycle (in particular during while the camera integrates an image, i.e. records a frame) where all the light sources are switched on. This is in contrast to RGB-projector-systems, where the color channels are projected consecutively, i.e. where only one color channel is active at a time—in such systems, balancing is achieved more easily.

Advantageously, imperfect balancing is used, which allows to carry out the balancing in a shorter time. Hence, advantageously, t1/t2 is larger than 0.6 or smaller than 0.4.

Brightness and/or Color Monitoring

Illumination device 9 may further comprise at least one light sensor for monitoring the brightness of the light source(s).

Such a sensor allows to monitor and control the brightness of the light sources 22a-22d as their efficiency changes with temperature and depending on the age of the light sources.

In particular, control unit 32 can be adapted to monitor the brightness of the light sources for generating calibration parameters and to use these calibration parameters for controlling the light sources, e.g. by adjusting the currents to the light sources.

For example, and as shown in FIG. 2, there may be one such light sensor 58a, 58b, 58c, 58d attributed to each one light source 22a, 22b, 22c, 22d, which is positioned to receive part of the light from its light source and to generate a signal indicative of its brightness.

Advantageously, however, there may be a single light sensor 58 positioned to measure light from all of several light sources 22a-22d. This can e.g. be achieved by a sensor 58 located at a position where the light from all light sources 22a-22d is combined, e.g. by using the spurious reflection of dichroic beam splitter 42c, which is the last dichroic beam splitter. It is optimized to cast all light towards spatial light modulator 24. However, since it is, in reality, not an ideal dichroic beam splitter, a small fraction of the light from each light source is reflected out of the optical axis of the illumination device and can be used for monitoring the brightness by means of light sensor 58.

In this embodiment, in order to individually measure the brightness of the individual light sources, control unit 32 can be equipped to perform calibration measurements outside the integration phase of camera 16. Such a calibration measurements may comprise the steps of
Placing spatial light modulator 24 into a non-transmitting configuration.
Switching on exactly one of the light sources 22a-22d and measuring a brightness signal for this light source by means of light sensor 58.

This scheme is, as mentioned, best performed when the device comprises a light sensor 58 positioned to measure light from all of several light sources. In particular, this light sensor is arranged to receive off-axial light from a last dichroic beam splitter used for combining the light of the light sources.

Such calibration phases are carried out for all light sources sequentially, either in the same frame cycle or in separate frame cycles of camera 16.

The calibration phases may e.g. be inserted during, immediately before or immediately after operating phases I2 or I4.

Optionally, the patterns in configurations I2 and I4 may be adapted to compensate for the time the mirrors are spending in their off-position during the calibration phases in order to maintain the condition of Eq. (1).

This kind of monitoring is particularly useful when the illumination device comprises several light sources 22a-22d of different colors, such as red, green, and blue LEDs. In this case, the brightness of each one of them can be monitored, which allows to adjust the relative brightness values of the light sources in order to maintain a desired spectral composition (i.e. color tone, such as a desired color temperature of normally white light), advantageously in a control loop.

Hence, advantageously, the light sources 22a-22d have different colors (i.e. their center wavelength differ by at least 20 nm). In that case, the method performed by the control unit of the device may comprise the step of using the calibration measurements for the light sources 22a-22d in order to maintain a desired relative brightness between at least two of the light sources 22a-22d when said at least two light sources are simultaneously switched on while recording a frame by means of the camera.

Notes

In the embodiments above, illumination device 9 comprises several individual light sources 22a-22d, e.g. two, three, four or even more light sources. It may, however, also comprise only a single light source, such as a white LED.

In the first aspect of the invention, camera 16 generates the master signal for triggering illumination device 9, i.e. the signal “camera synch” in FIG. 3 is generated by camera 16. In other implementations of the invention, however, camera 16 may also operate in triggered mode, i.e. it integrates a frame upon receiving an external trigger. In that case, control unit 32 may e.g. generate the trigger signal, triggering the recording of a frame by means of camera 16, i.e. the signal “camera synch” in FIG. 4 may not be generated by camera 16 but by control unit 32.

While there are shown and described presently preferred embodiments of the invention, it is to be distinctly understood that the invention is not limited thereto but may be otherwise variously embodied and practiced within the scope of the following claims.

1. An ophthalmologic microscope comprising
i) an illumination device generating illumination pulses and having
a) at least one light source,
b) a spatial light modulator, and
c) illumination imaging optics,
ii) a microscope device having
a) microscope optics and
b) at least one electronic camera, and
iii) a control unit,
wherein said camera has a frame signal output carrying a signal indicative of times when the camera, records a frame and
wherein said control unit is structured to synchronize said illumination device to said frame signal output.

2. An ophthalmologic microscope comprising
i) an illumination device generating illumination pulses and having
a) at least one light source,
b) a spatial light modulator, and
c) illumination imaging optics,
ii) a microscope device having
a) microscope optics and
b) at least one electronic camera, and
iii) a control unit,
wherein said control unit is structured to generate at least two illumination pulses for each frame recorded by said camera.

3. The microscope of claim 1, wherein said camera is configured to operate in free-running mode.

4. The microscope of claim 1, wherein said spatial light modulator is an electronically controlled spatial light modulator comprising a two-dimensional array of individually controllable pixels, and wherein said control unit is structured to synchronize a state of said pixels to said frame signal output.

5. The microscope of claim 4, wherein said spatial light modulator comprises a two-dimensional array of micro-mirrors individually deflectable into a first and a second position.

6. The microscope of claim 5, wherein said control unit is structured to bring, for a group of no more than N consecutive frame cycles of said camera, each micro-mirror of said spatial light modulator into the first position during a time t1 and into the second position during a time t2, wherein
0.1<t1/t2<10,
wherein N≤100.

7. The microscope of claim 1, wherein said control unit is structured to pulse said light source in a manner synchronized to said frame signal output.

8. The microscope of claim 7,
wherein said spatial light modulator is an electronically controlled spatial light modulator comprising a two-dimensional array of individually controllable pixels, and wherein said control unit is structured to synchronize a state of said pixels to said frame signal output and
wherein the control unit is structured to
bringing the pixels into a given configuration during a dark phase prior to a given light pulse, and
starting the light pulse only when the pixels are in the given configuration.

9. The microscope of claim 7, wherein said control unit is structured to generate, for each frame recorded by said camera, at least two separate light pulses, wherein one of the light pulses falls into the integration phase of the camera while the other light pulse(s) fall outside the integration phase.

10. The microscope of claim 9, wherein
the light pulses have equal durations,
for all light pulses, the spatial light modulator has the same configurations, and
the light pulses are separated by dark phases of equal length.

11. The microscope of claim 10, wherein said control unit is adapted to bring, between the light pulses the spatial light modulator into configurations opposite to the configurations during the light pulses.

12. The microscope of claim 1, wherein said control unit is adapted to
predicting a time when said camera will record a next frame depending on the time when the camera recorded at least one previous frame, and
preparing at least part of said illumination device before the predicted time for recording the next frame.

13. The microscope of claim 1, further comprising at least one light sensor adapted to measure a light intensity between said light source and said spatial light modulator, wherein said control unit is structured to
bringing said light modulator into a non-transmitting mode, and
while said light modulator is in said non-transmitting mode, pulsing said at least one light source for measuring a brightness of said light source.

14. The microscope of claim 1, further comprising at least one light sensor for monitoring a brightness of the light source, wherein said control unit is adapted to monitor the brightness of the light source by light sensor for generating calibration parameters and to use the calibration parameters for controlling the at least one light source.

15. The microscope of claim 14, comprising several light sources, wherein said control unit is adapted to carry out calibration measurements outside an integration phase of said camera, wherein said calibration measurements comprise;
placing said spatial light modulator into a non-transmitting configuration and
switching on exactly one of the light sources and measuring a brightness signal by the light sensor.

16. A method for operating an ophthalmologic microscope, wherein said device comprises
i) an illumination device having
a) at least one light source,
b) a spatial light modulator, and
c) illumination imaging optics,
ii) a microscope device having
a) microscope optics and
b) at least one electronic camera, and
iii) a control unit,
wherein said camera has a frame signal output carrying a signal indicative of times when the camera records a frame and
wherein said method comprises;
sequentially recording frames by said camera and
generating, by means of the illumination device, light pulses synchronized to said frame signal output.

17. A method for operating an ophthalmologic microscopes wherein said device comprises
i) an illumination device generating illumination pulses and having
a) at least one light source,
b) a spatial light modulator, and
c) illumination imaging optics,
ii) a microscope device having
a) microscope optics and
b) at least one electronic camera, and
a control unit,
wherein said method comprises;
sequentially recording frames said camera and
generating, by said illumination device, at least two illumination pulses for each frame recorded by said camera.

18. The method of claim 16, wherein said camera is run in free-running mode.

19. The method of claim 16, said spatial light modulator is an electronically controlled spatial light modulator comprising a two-dimensional array, wherein said pixels are controlled individually, and wherein said method comprises controlling the pixels in a manner synchronized to said frame signal output.

20. The method of claim 19, wherein said spatial light modulator comprises a two-dimensional array of micro-mirrors, wherein said micro-mirrors are individually deflected into a first and a second position.

21. The method of claim 20, comprising, for each frame recorded by said camera, bringing each micro-mirror of said spatial light modulator into the first position during a time t1 and into the second position during a time t2, wherein t1 and t2 are equal.

22. The method of claim 16, comprising pulsing said light source in a manner synchronized to said frame signal output.

23. The method of claim 22, wherein said spatial light modulator is an electronically controlled spatial light modulator comprising a two-dimensional array, wherein said pixels are controlled individually,
wherein said method comprises;
controlling the pixels in a manner synchronized to said frame signal output,
bringing the pixels into a given configuration during a dark phase prior to a given light pulse, and
starting the light pulse only when the pixels are in the given configuration.

24. The method of claim 22, comprising generating, for each frame recorded by said camera, at least two separate light pulses, wherein one of the light pulses falls into the integration phase of the camera while the other light pulse(s) fall outside the integration phase.

25. The method of claim 24, wherein
the light pulses have equal durations,
for all light pulses, the spatial light modulator has the same configurations, and
the light pulses are separated by dark phases of equal length.

26. The method of claim 25, comprising bringing, between the light pulses, the spatial light modulator into configurations opposite to the configurations during the light pulse.

27. The method of claim 16, comprising;
predicting a time when said camera will record a next frame depending on the time when the camera recorded at least one previous frame, and
preparing at least part of said illumination device before the predicted time for recording the next frame.

28. The method of claim 16, wherein said device further comprises at least one light sensor adapted to measure a light intensity between said light source and said spatial light modulator,
wherein said method comprises;
bringing said light modulator into a non-transmitting mode, and
while said light modulator is in said non-transmitting mode, pulsing said at least one light source for measuring a brightness of said light source.

29. The method of claim 16, wherein said microscope comprises at least one light sensor and comprising:
monitoring a brightness of the light source by means of said light sensor and generating calibration parameters and
using the calibration parameters for controlling the light source.

30. The method of claim 29, wherein said microscope comprises several light sources, wherein said method comprises calibration measurements outside an integration phase of said camera, wherein said calibration measurements comprise:
placing said spatial light modulator into a non-transmitting configuration and
switching on exactly one of the light sources and measuring a brightness signal by the light sensor.

31. The method of claim 30, wherein said light sources have different colors and wherein said method further comprises using the calibration measurements for the light sources in order to maintain a desired relative brightness between at least two of the light sources when said two light sources are simultaneously switched on while recording a frame.
